(19) United States
(12) Patent Application Publication
(10) Pub. No.: US 2006/0070549 A1
(43) Pub. Date: Apr. 6, 2006

(54) Self-dispersible colorant and ink composition containing the same

(75) Inventors: Yeon-kyoung Jung, Suwon-si (KR); Seung-min Ryu, Yongin-si (KR)

Correspondence Address: STAAS & HALSEY LLP, SUITE 700, 1201 NEW YORK AVENUE, N.W., WASHINGTON, DC, 20005, US

(73) Assignee: SAMSUNG ELECTRONICS CO., LTD., Suwon-si (KR)

(21) Appl. No.: 11/239,060

(22) Filed: Sep. 30, 2005

(30) Foreign Application Priority Data
Oct. 1, 2004 (KR) 10-2004-0078269

(51) Int. Cl. C09D 11/00; C08K 5/00; C07D 403/02

(52) U.S. Cl. 106/31.47; 106/31.77; 106/31.48; 106/31.58; 106/31.86; 106/498; 106/493; 544/188; 534/635

(57) ABSTRACT

A self-dispersing colorant containing a triazine moiety and an ink composition including the same are disclosed. The ink composition has excellent storage ability. Further, images formed using the ink composition have excellent light fastness and excellent water resistance.

### CROSS-REFERENCE TO RELATED APPLICATIONS

This application claims the benefit of Korean Patent Application No. 10-2004-0078269, filed on Oct. 1, 2004, in the Korean Intellectual Property Office, the disclosure of which is incorporated herein in its entirety by reference.

### BACKGROUND OF THE INVENTION

1. Field of the Invention

The present invention relates to a self-dispersible colorant and an ink composition containing the same, and more particularly, to a self-dispersible colorant with excellent light fastness and excellent water resistance due to a triazine moiety contained therein, and an ink composition containing the same.

2. Description of the Related Art

Inkjet printing is non-impact printing. Compared to impact printing, inkjet printing is much quieter. Further, by using inkjet printing, color images can easily form compared to when laser-beam printing is used.

The non-impact printing is classified into continuous inkjet systems and drop-on demand (DOD) systems. In the continuous inkjet systems, when ink is emitted in a continuous stream, the direction of the ink emitted is adjusted by changing an electromagnetic field. In DOD systems, ink is broken up into droplets, and the droplets are expelled. DOD systems are classified into thermal-bubble inkjet systems and piezoelectric inkjet systems. The thermal-bubble inkjet systems use the pressure produced by bursting bubbles of the heated ink to fire the ink. In piezoelectric systems, electricity passing through piezo elements causes physical distortions in the elements, which helps to fire the ink.

Recently, inkjet printers are required to have much smaller dot sizes, and also to produce a developed product with high quality at high resolution. In order to obtain smaller dot sizes, a head of an inkjet printer has a much smaller nozzle opening. Therefore, such a nozzle opening is very likely to be clogged, and sediments or the like affects the performance of the inkjet printer because they in part determine the size of the inkjet droplets. Meanwhile, it is well known that an ink composition is in part responsible for the nozzle clogging. Therefore, in order to solve the clogging, a humectant is commonly added to the ink composition.

The ink composition for inkjet printing is basically composed of a colorant used to give color, a solvent, and an additive. When the colorant is a dye, light fastness and water resistance deteriorate although various colors can be produced.

On the other hand, when the colorant is a pigment, a polymer dispersing agent containing a hydrophilic group and a hydrophobic group is commonly used with the pigment. The hydrophobic group contained in the polymer dispersing agent reacts with the colorant to stabilize the dispersion of the colorant. The hydrophilic group contained in the polymer dispersing agent reacts with an aqueous solution to induce steric stability.

However, even when an amount of the polymer dispersing agent increases a little, physical properties of the ink composition such as viscosity change substantially because the polymer dispersing agent has a large molecular weight. Therefore, it is very difficult to adjust the amount of the polymer dispersing agent. In addition, although the polymer dispersing agent contains the hydrophilic group, the fraction of the hydrophilic group is too small with respect to the polymer. Therefore, it takes a long time to completely dissolve the ink composition.

Meanwhile, the pigment has high light fastness compared to the dye. However, when the pigment is exposed to ultraviolet (UV) rays, including the sun's ray, it can be decolored or discolored. Therefore, a light fastness-improving agent is further added to increase light fastness when the dye and the pigment are used. For example, U.S. Pat. No. 6,346,595 discloses a silicon-containing compound used as the light fastness-improving agent. The silicon-containing compound is used to increase a UV-blocking effect in the ink composition. In this case, however, the silicon-containing compound has a complex structure, and does not mix well with the other components of the ink composition when the silicon-containing compound is heavy. That is, the addition of the light fastness-improving agent facilitates nozzle clogging due to an increase in the cohesion of ink, and decreases in the homogeneity of the ink composition.

### SUMMARY OF THE INVENTION

Additional aspects and/or advantages of the invention will be set forth in part in the description which follows and, in part, will be apparent from the description, or may be learned by practice of the invention.

The present invention provides a colorant capable of self-dispersing without a dispersing agent, the colorant has excellent light resistance and excellent water resistance.

The present invention also provides an ink composition containing the self-dispersing colorant.

According to an aspect of the present invention, there is provided a self-dispersing colorant represented by formula 1: 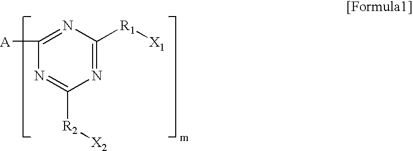
, where A is a colorant residue;
R₁ and R₂ are each independently chemical bonding, a substituted or unsubstituted C1-C10 alkylene group, a substituted or unsubstituted C6-C10 arylene group, a substituted or unsubstituted C2-C10 heteroarylene group, or a substituted or unsubstituted C4-C10 cycloalkylene group;
X₁ and X₂ are each independently a carboxylic acid group, a salt thereof, a sulfonic acid, a salt thereof, or an amino group; and
m is an integer between 1 and 10.

According to an aspect of the present invention, an ink composition is provided to include the self-dispersing agent and an aqueous liquid medium.

### DETAILED DESCRIPTION OF THE PREFERRED EMBODIMENTS

Reference will now be made in detail to the embodiments of the present invention wherein like reference numerals refer to the like elements throughout. The embodiments are described below to explain the present invention.

A self-dispersing agent according to an embodiment of the present invention is represented by formula 1: 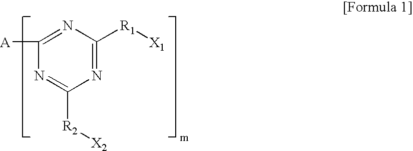
, where A is a colorant residue;
R₁ and R₂ are each independently chemical bonding, a substituted or unsubstituted C1-C10 alkylene group, a substituted or unsubstituted C6-C10 arylene group, a substituted or unsubstituted C2-C10 heteroarylene group, or a substituted or unsubstituted C4-C10 cycloalkylene group;
X₁ and X₂ are each independently a carboxylic acid group, a salt thereof, a sulfonic acid, a salt thereof, or an amino group; and
m is an integer between 1 and 10.

In formula 1, the colorant residue A is a residue derived from dye or a pigment that is commonly used in a conventional ink composition. That is, the dye and the pigment each must contain an active site (an amino group, a carboxy group, a hydroxy group, or sulfonic acid group, or the like) to which a triazine compound is bonded, or can form such an active site therein through other reactions. Examples of the dye include C.I Direct Black 9, 17, 19, 22, 32, 56, 91, 94, 97, 166, 168, 174, and 199; C.I Direct Blue 1, 10, 15, 22, 77, 78, 80, 200, 201, 202, 203, 207, and 211; C.I Direct Red 2, 4, 9, 23, 31, 39, 63, 72, 83, 84, 89, 111, 173, 184, and 240; C.I Direct Yellow 8, 9, 11, 12, 27, 28, 29, 33, 35, 39, 41, 44, 50, 53, and 58; and the like, but are not limited thereto. Examples of the pigment include a carbon black, graphite, a vitreous carbon, an activated charcoal, an activated carbon, anthraquinone, a phthalocyanine blue, a phthalocyanine green, diazos, monoazos, pyranthrones, perylene, quinacridone, indigoid pigments, and the like, but are not limited thereto.

The substituted or unsubstituted C1-C10 alkylene group in formula 1 may be an ethylene group, or the like. The substituted or unsubstituted C6-C10 arylene group in formula 1 may be a phenylene group, or the like. The substituted or unsubstituted C2-C10 heteroarylene group in formula 1 may be a thiazolylene group, or the like. The substituted or unsubstituted C4-C10 cycloalkylene group may be a cyclohexylene group, or the like.

A method of manufacturing a self-dispersing colorant will now be described.

A dye or pigment commonly used is reacted with a triazine compound, such as cyanuric chloride (also known as 2,4,6-trichloro-1,3,5-triazine). In this case, an active site, such as an amino group, a hydroxy group, or the like, contained in the dye or pigment is bonded to the triazine compound. Then, a substituent of the triazine cycle contained in the result is substituted by a hydrophilic group (or an ionic group), including a carboxylic acid group, a salt thereof, a sulfonic acid group, and a salt thereof.

The self-dispersing colorant, in particular when a colorant residue is the pigment, can self-disperse by the substituted hydrophilic group in the triazine cycle. That is, the self-dispersing colorant is not required to have a dispersing agent. In addition, the triazine cycle contained in the self-dispersing colorant improves the light fastness of the colorant.

An ink composition containing a self-dispersing colorant will now be described.

The ink composition according to an embodiment of the present invention includes an aqueous liquid medium, and the self-dispersing colorant represented by formula 1. At this time, an amount of the self-dispersing colorant may be in the range of 1 to 40 parts by weight based on 100 parts by weight of the aqueous liquid medium. When the amount of the self-dispersing colorant is outside this range, the physical properties of the ink composition deteriorate, which is undesirable.

The self-dispersing colorant may be compounds represented by formula 2 through 4:

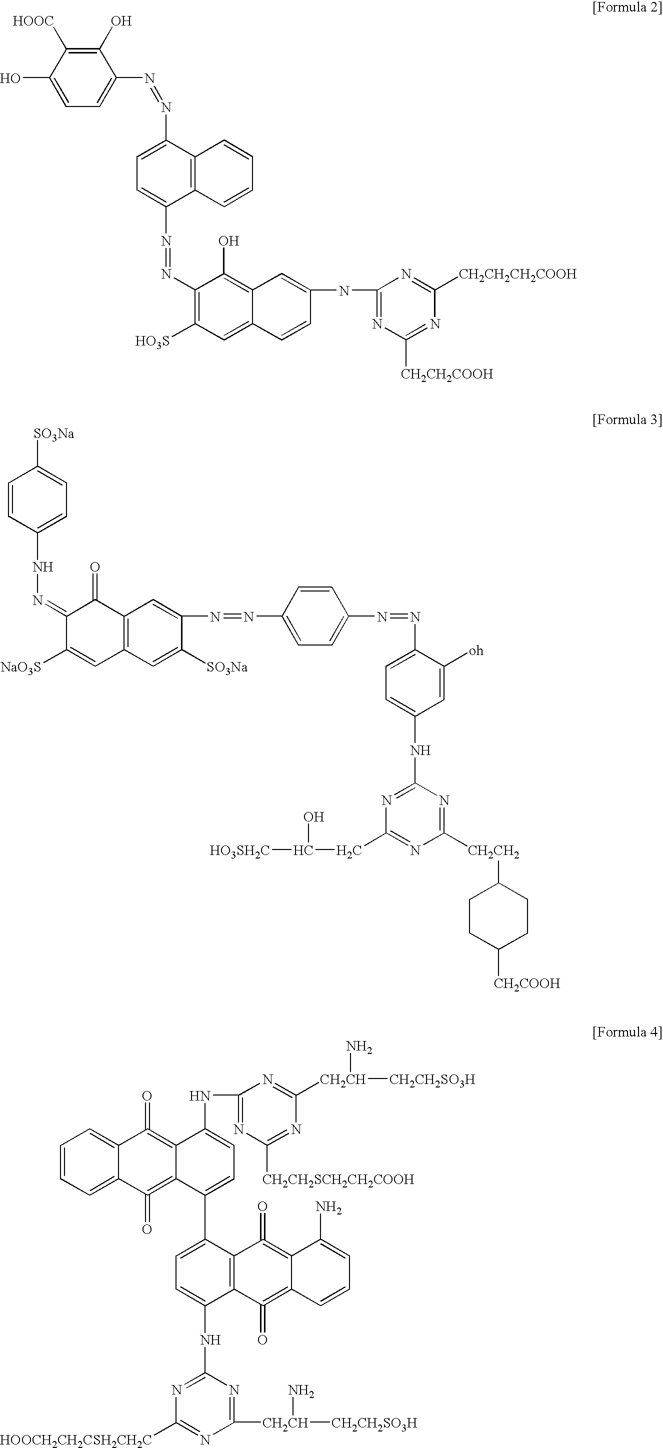

The ink composition according to the present embodiment may further include a commonly used colorant, in addition to the self-dispersing colorant represented by formula 1. At this time, an amount of the commonly used colorant may be in the range of 5 to 1500 parts by weight based on 100 parts by weight of the self-dispersing colorant represented by formula 1.

Ink compositions according to an embodiment and another embodiment of the present invention each include the commonly used colorant and the self-dispersing colorant dissolved or dispersed in the aqueous liquid medium.

The aqueous liquid medium may be water alone, or a mixture of an organic solvent and water. In the latter case, an amount of the organic solvent may be in the range of 5 to 50 parts by weight, and an amount of water may be in the range of 50 to 95 parts by weight.

At this time, the amounts of the organic solvent and water may vary according to the characteristics of the ink composition, such as viscosity, surface tension, dry speed, or the like. Further, those characteristics of the ink composition are determined in part by an inkjet printing method using ink, or equipment by which ink is printed, or the like.

The organic solvent may be alcohols, including methylalcohol, ethylalcohol, n-propylalcohol, isopropylalcohol, n-butylalcohol, sec-butylalcohol, t-butylalcohol, isobutylalcohol, and the like; ketones, including acetone, methylethylketone, diacetone alcohol, and the like; esters, including ethyl acetate, ethyl lactate, and the like; polyhydric alcohols, including ethyleneglycol, diethyleneglycol, triethyleneglycol, propyleneglycol, butyleneglycol, 1,4-butanediol, 1,2,4-butanetriol, 1,5-pentanediol, 1,2,6-hexanetriol, hexyleneglycol, glycerol, glycerol ethoxylate, trimethylolpropane ethoxylate, and the like; lower alkyl ethers, including ethyleneglycol monomethyl ether, ethyleneglycol monoethyl ether, diethyleneglycol methyl ether, diethyleneglycol ethyl ether, triethyleneglycol monomethyl ether, triethyleneglycol monoethyl ether, and the like; nitrogen-containing compounds, including 2-pyrrolidone, N-methyl-2-pyrrolidone, and the like; and sulfur-containing compounds, including dimethyl sulfoxide, tetramethylenesulfone, thioglycol, and the like.

The ink composition may further include a humectant such that the ink composition is not clogged in a nozzle. The humectant may be a polyhydric alcohol. In detail, the humectant may be glycerin, ethyleneglycol, diethyleneglycol, triethyleneglycol, propyleneglycol, dipropyleneglycol, hexyleneglycol, 1,3-butanediol, 1,4-butanediol, 1,5-pentanediol, 2-buten-1,4-diol, 2-methyl-2-pentanediol, or mixtures of these.

Ink compositions according to an embodiment and another embodiment of the present invention each may include 5 to 40 parts by weight of the humectant based on 100 parts by weight of the aqueous liquid medium.

The ink composition may further include a surfactant to stabilize the jetting performance of ink from a nozzle by adjusting the surface tension of the ink composition, and to control an amount of ink fired onto a medium. The surfactant may be an anionic surfactant, a cationic surfactant, or a non-ionic surfactant. The anionic surfactant may be C1-1000 alkylcarboxylic acid salts (preferably, C10-200 alkylcarboxylic acid salts), C1-1000 alcohol sulfonic acid ester salts (preferably, C10-200 alcohol sulfonic acid ester salts), C1-1000 alkylsufonic acid salts (preferably, C10-200 alkylsufonic acid salts), C1-1000 alkylbenzenesulfonic acid salts (preferably, C10-200 alkylbenzenesulfonic acid salts), or mixtures of these. The cationic surfactant may be salts of a fatty acid amine, quaternary ammonium salts, sulfonium salts, phosphonium salts, or mixtures of these. The non-ionic surfactant may be polyoxyethylene alkyl ether (wherein alkyl is a C1-1000 alkyl group, preferably a C10-200 alkyl group), polyoxyethylene alkyl phenyl ether (wherein alkyl is a C1-1000 alkyl group, preferably a C10-200 alkyl group), polyoxyethylene secondary alcohol ether, polyoxyethylene-oxypropylene block copolymer, polyglycerin fatty acid ester, sorbitan fatty acid ester, or mixtures of these. The ink composition according to the present embodiment may include 0.1 to 10 parts by weight of the surfactant based on 100 parts by weight of the aqueous liquid medium.

The ink composition may further include a viscosity-adjusting agent. The viscosity-controlling agent adjusts the viscosity of the ink composition such that the ink is smoothly expelled from the nozzle. The viscosity-adjusting agent may be casein, hydroxymethylcellulose, hydroxyethylcellulose, carboxymethylcellulose, or the like, but is not limited thereto. The amount of the viscosity-adjusting agent may be in a range of 0.1 to 10 parts by weight based on 100 parts by weight of the aqueous liquid medium.

In addition, the ink composition may further include a pH adjusting agent, an antioxidant, or the like.

A method of manufacturing the ink composition will now be described.

First, a self-dispersing colorant according to the present embodiment is mixed with water. Next, an organic solvent, a humectant, a surfactant, a viscosity-adjusting agent, or the like is added to the result. Then, the result mixture is stirred to obtain a homogeneous solution.

Finally, the homogeneous solution was filtered to obtain an ink composition according to the present embodiment.

The present invention will be described in more detail by presenting the following examples. These examples are for illustrative purposes only, and are not intended to limit the scope of the present invention.

### EXAMPLE 1

| | | |
|---|---|---|
| The self-dispersing colorant represented by formula 2 | 4 | g |
| Water | 77 | g |
| Isopropyl alcohol | 3 | g |
| Ethylene glycol | 10 | g |
| Glycerine | 6 | g |

The above-components were mixed for at least 30 minutes to obtain a homogenous solution. Then, the result was filtered through a 0.45 μm filter to manufacture an ink composition according to the present invention.

### EXAMPLE 2

An ink composition was manufactured in the same manner as in Example 1, except that the self-dispersing colorant represented by formula 3 was used instead of the self-dispersing colorant represented by formula 2.

### EXAMPLE 3

An ink composition was manufactured in the same manner as in Example 1, except that the self-dispersing colorant represented by formula 4 and a 0.80 μm filter were used instead of the self-dispersing colorant represented by formula 2 and the 0.45 μm filter, respectively.

### COMPARATIVE EXAMPLE 1

An ink composition was manufactured in the same manner as in Example 1, except that C.I. Direct Black 51 was used instead of the self-dispersing colorant represented by formula 2.

### COMPARATIVE EXAMPLE 2

An ink composition was manufactured in the same manner as in Comparative Example 1, except that Acid Black 191 was used instead of C.I. Direct Black 51.

### COMPARATIVE EXAMPLE 3

An ink composition was manufactured in the same manner as in Comparative Example 1, except that Pigment Red 177 was used instead of C.I. Direct Black 51, 4 g of joncryl 61 (35% aqueous solution, Joncryl Polymer) was used as an dispersing agent, the amount of water was 73 g, and a 0.80 μm filter was used instead of the 0.45 μm filter.

The characteristics of the ink compositions manufactured in the above Examples 1 through 3 and Comparative Examples 1 through 3 were measured according to the following methods.

(1) Test of Storage Ability

100 ml of the ink compositions obtained in Examples 1 through 3 and Comparative Examples 1 through 3 were respectively placed in heat resistant glass bottles, sealed and then stored at 60° C. in a constant temperature bath. The bottles were left for 2 months and then it was confirmed whether or not precipitates were formed at the bottoms of the bottles. The results are shown in Table 1.

o: no precipitate x: precipitate

TABLE 1

| | Example | | | Comparative example | | |
|---|---|---|---|---|---|---|
| | 1 | 2 | 3 | 1 | 2 | 3 |
| Storage ability | ◯ | ◯ | ◯ | ◯ | ◯ | ◯ |

Referring to Table 1, the ink compositions obtained in Examples 1 through 3 and Comparative Examples 1 through 3 did not precipitate. Based on such results, it was confirmed that both sets of ink compositions, these obtained in Examples 1 through 3 and those obtained in Comparative Examples 1 through 3 have excellent long-term storage abilities.

(2) Test of Nozzle Clogging

The ink compositions obtained in Examples 1-3 and Comparative Examples 1-3 were placed in Samsung ink cartridges. At this time, the Samsung ink cartridges were not capped. Then, the uncapped Samsung ink cartridges were left at room temperature for 1 month. The resultant cartridges were respectively placed in a printer, and then a nozzle was cleaned to evaluate the degree of nozzle clogging. The results are shown in Table 2.

O: a nozzle was normalized after being cleaned 5 times or less

X: a nozzle was not normalized after being cleaned 6 times or more

TABLE 2

| | Example | | | Comparative example | | |
|---|---|---|---|---|---|---|
| | 1 | 2 | 3 | 1 | 2 | 3 |
| Nozzle clogging | ◯ | ◯ | ◯ | ◯ | ◯ | X |

When the ink compositions obtained in Examples 1-3 were used, the nozzles were not clogged. Therefore, it was confirmed that the ink compositions obtained in Examples 1-3 have excellent storage ability in the cartridges. On the other hand, when the ink composition including the commonly used pigment and the dispersing agent obtained in Comparative Examples 3 was used, the nozzle was clogged.

(3) Test of Water Resistance

The ink compositions obtained in Examples 1-3 and Comparative Examples 1-3 were placed in Samsung ink cartridges, and then a solid pattern 2 cm×2 cm was printed using each ink composition. The printed images were dried for one hour, dipped in a bottle filled with distilled water for 5 minutes, and then dried again. The changes of OD values before and after dipping into the distilled water were determined. The results are shown in Table 3.

o: the change is less than 20% with respective to the initial OD

x: the change is at least 20% with respect to the initial OD

TABLE 3

| | Example | | | Comparative example | | |
|---|---|---|---|---|---|---|
| | 1 | 2 | 3 | 1 | 2 | 3 |
| Water resistance | ◯ | ◯ | ◯ | X | X | ◯ |

Referring to Table 3, the ink compositions obtained in Examples 1 and 2 had superior water resistance compared to the ink compositions obtained in Comparative Examples 1 and 2.

(4) Test of Light Fastness

The ink compositions obtained in Examples 1-3 and Comparative Examples 1-3 were placed in Samsung ink cartridges, and then a solid pattern 2 cm×2 cm was printed using each ink composition. The printed images were exposed to light in a Q-SUN Xenon Test Chamber for 100 hours. Subsequently, the changes in OD values before and after exposure were determined and estimated as follows. The results are shown in Table 4.
A=OD (after exposure)/OD (before exposure)×100(%)

O: A≧90%

Δ: 75≦A<90%

X: A<75%

TABLE 4

| | Example | | | Comparative example | | |
|---|---|---|---|---|---|---|
| | 1 | 2 | 3 | 1 | 2 | 3 |
| Light fastness | ◯ | ◯ | ◯ | X | X | Δ |

Referring to Table 4, the ink compositions obtained in Examples 1 and 2 had superior light fastness compared to the ink compositions obtained in Comparative Examples 1 and 2.

A self-dispersing colorant represented by formula 1 according to the present invention can be dissolved or dispersed in an aqueous solution without an additional dispersing agent. An ink composition including the self-dispersing colorant has excellent storage ability. Further, an image formed using the ink composition has excellent light fastness and excellent water resistance.

While the present invention has been particularly shown and described with reference to exemplary embodiments thereof, it will be understood by those of ordinary skill in the art that various changes in form and details may be made therein without departing from the spirit and scope of the present invention as defined by the following claims.

Although a few embodiments of the present invention have been shown and described, it would be appreciated by those skilled in the art that changes may be made in these embodiments without departing from the principles and spirit of the invention, the scope of which is defined in the claims and their equivalents.

What is claimed is:

1. A self-dispersing colorant represented by formula 1: 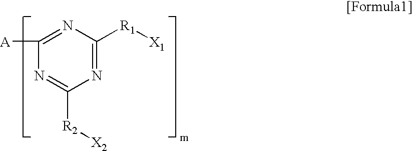
, where A is a colorant residue;
R₁ and R₂ are each independently chemical bonding, a substituted or unsubstituted C1-C10 alkylene group, a substituted or unsubstituted C6-C10 arylene group, a substituted or unsubstituted C2-C10 heteroarylene group, or a substituted or unsubstituted C4-C10 cycloalkylene group;
X₁ and X₂ are each independently a carboxylic acid group, a salt thereof, a sulfonic acid, a salt thereof, or an amino group; and
m is an integer between 1 and 10.

2. The self-dispersing colorant of claim 1, wherein A is a residue derived
from a pigment selected from the group consisting of C.I Direct Black 9, 17, 19, 22, 32, 56, 91, 94, 97, 166, 168, 174, and 199; C.I Direct Blue 1, 10, 15, 22, 77, 78, 80, 200, 201, 202, 203, 207, and 211; C.I Direct Red 2, 4, 9, 23, 31, 39, 63, 72, 83, 84, 89, 111, 173, 184, and 240; C.I Direct Yellow 8, 9, 11, 12, 27, 28, 29, 33, 35, 39, 41, 44, 50, 53, and 58; carbon black; graphite; a vitreous carbon; an activated charcoal; an activated carbon; anthraquinone; a phthalocyanine blue; a phthalocyanine green; diazos; monoazos; pyranthrones; perylene; quinacridone; and indigoid pigments.

3. The self-dispersing colorant of claim 1, represented by one of formulas 2 through 4:

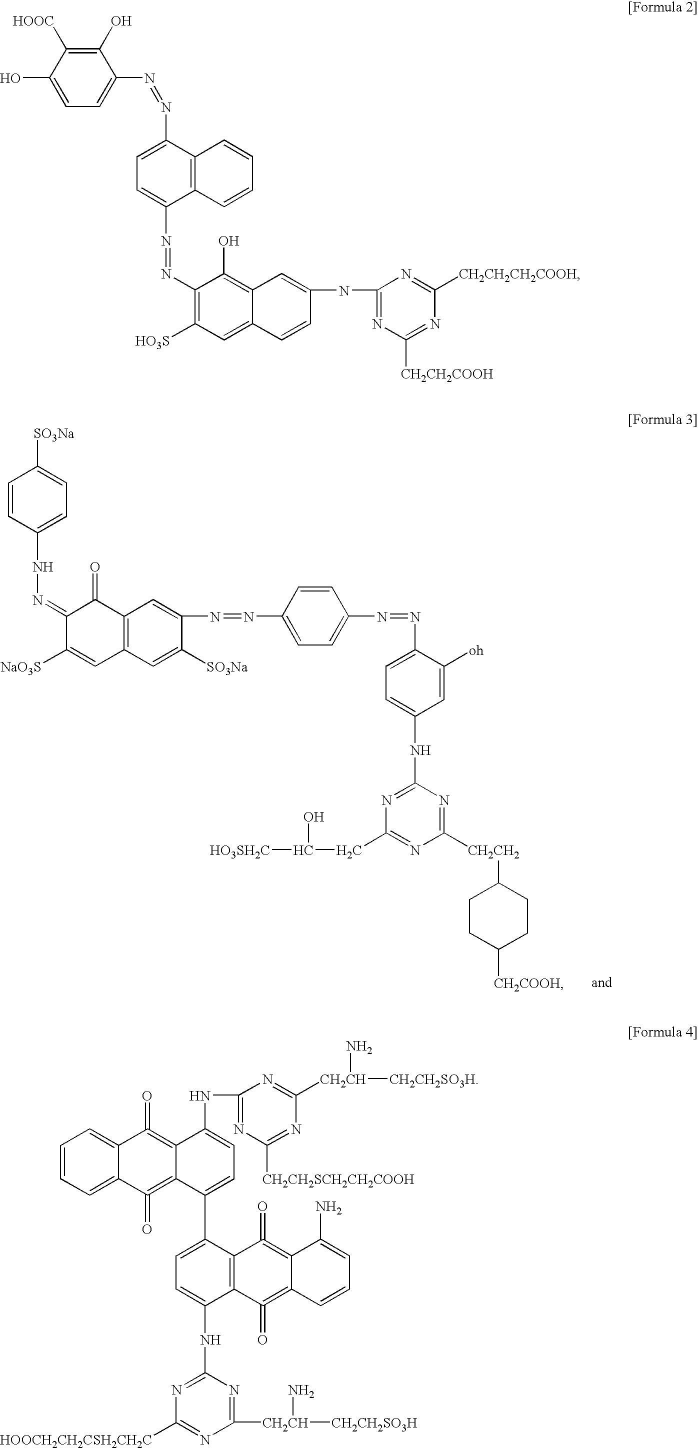

4. An ink composition comprising a liquid medium and a self-dispersing colorant represented by formula 1: 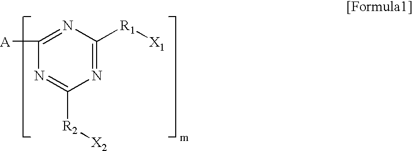
, where A is a colorant residue;
R₁ and R₂ are each independently chemical bonding, a substituted or unsubstituted C1-C10 alkylene group, a substituted or unsubstituted C6-C10 arylene group, a substituted or unsubstituted C2-C10 heteroarylene group, or a substituted or unsubstituted C4-C10 cycloalkylene group;
X₁ and X₂ are each independently a carboxylic acid group, a salt thereof, a sulfonic acid, a salt thereof, or an amino group; and
m is an integer between 1 and 10.

5. The ink composition of claim 4, wherein an amount of the self-dispersing colorant is in the range of 1 to 40 parts by weight based on 100 parts by weight of the liquid medium.

6. The ink composition of claim 4, wherein the liquid medium is water, or a mixture of water and at least one organic solvent.

7. The ink composition of claim 4,
wherein the liquid medium is a mixture of organic solvent and water, and the organic solvent present in an amount of 5 to 50 parts by weight based on 100 parts by weight of the liquid medium.

8. The ink composition of claim 6, wherein the organic solvent is at least one solvent selected from the group consisting of methylalcohol, ethylalcohol, n-propylalcohol, isopropylalcohol, n-butylalcohol, sec-butylalcohol, t-butylalcohol, isobutylalcohol, acetone, methylethylketone, diacetone alcohol, ethyl acetate, ethyl lactate, ethyleneglycol, diethyleneglycol, triethyleneglycol, propyleneglycol, butyleneglycol, 1,4-butanediol, 1,2,4-butanetriol, 1,5-pentanediol, 1,2,6-hexanetriol, hexyleneglycol, glycerol, glycerol ethoxylate, trimethylolpropane ethoxylate, ethyleneglycol monomethyl ether, ethyleneglycol monoethyl ether, diethyleneglycol methyl ether, diethyleneglycol ethyl ether, triethyleneglycol monomethyl ether, triethyleneglycol monoethyl ether, 2-pyrrolidone, N-methyl-2-pyrrolidone, dimethyl sulfoxide, tetramethylenesulfone, and thioglycol.

9. The ink composition of claim 4, further comprising at least an additive selected from the group consisting of a viscosity-adjusting agent, a surfactant, a storage stabilizer, and a humectant.

10. The ink composition of claim 4, wherein A shown in formula 1 is a
residue derived from a pigment selected from the group consisting of C.I Direct Black 9, 17, 19, 22, 32, 56, 91, 94, 97, 166, 168, 174, and 199; C.I Direct Blue 1, 10, 15, 22, 77, 78, 80, 200, 201, 202, 203, 207, and 211; C.I Direct Red 2, 4, 9, 23, 31, 39, 63, 72, 83, 84, 89, 111, 173, 184, and 240; C.I Direct Yellow 8, 9, 11, 12, 27, 28, 29, 33, 35, 39, 41, 44, 50, 53, and 58; carbon black; graphite; a vitreous carbon; an activated charcoal; an activated carbon; anthraquinone; a phthalocyanine blue; a phthalocyanine green; diazos; monoazos; pyranthrones; perylene; quinacridone; and indigoid pigments.

11. The ink composition of claim 4, wherein the self-dispersing colorant is represented by one of formulas 2 through 4:

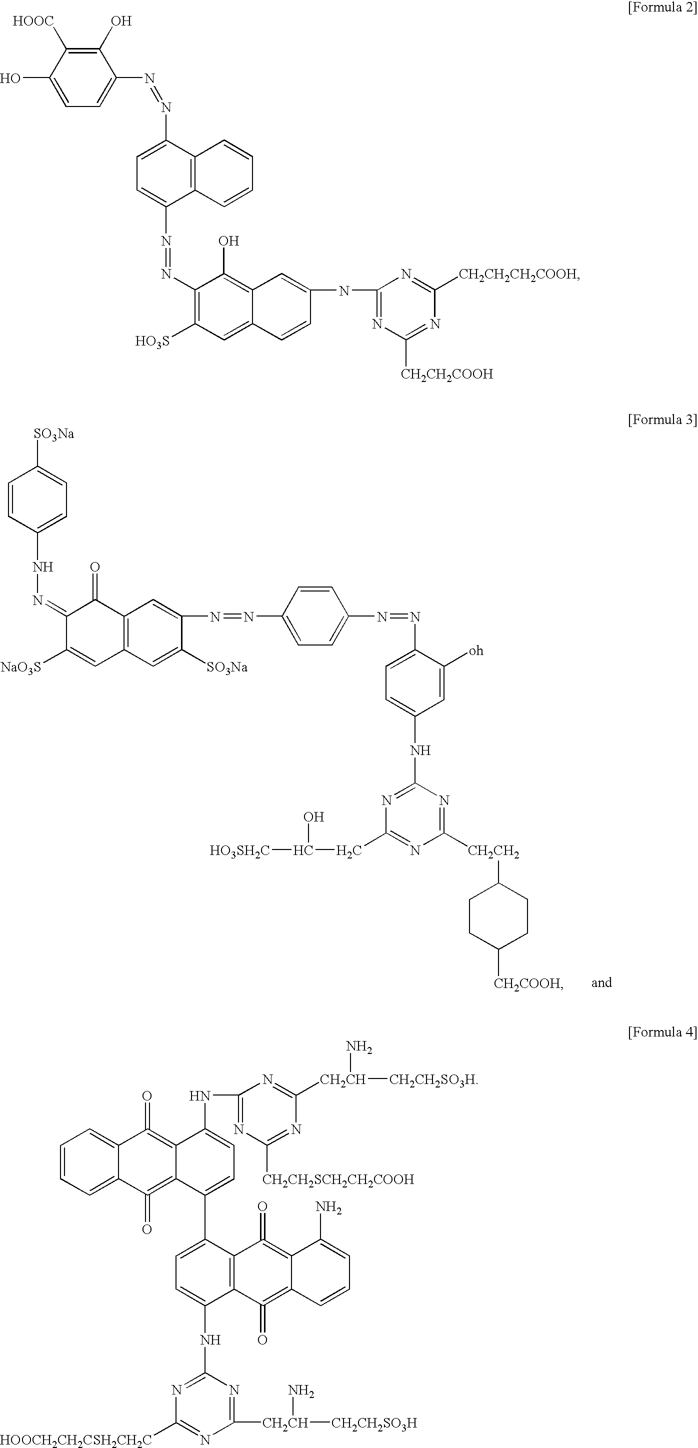

12. The ink composition of claim 6, wherein the organic solvent is at least one solvent selected from the group consisting of alcohols, ketones, esters, lower alkyl ethers, nitrogen-containing compounds, and sulfur-containing compounds.

13. A method of manufacturing an ink composition, comprising:
combining a liquid medium with a self-dispersing colorant represented by Formula 1: 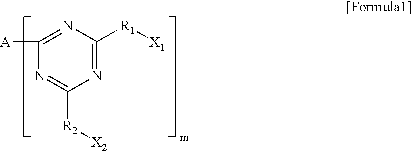
, where A is a colorant residue;
R₁ and R₂ are each independently chemically bonded a substituted or unsubstituted C1-C10 alkylene group, a substituted or unsubstituted C6-C10 arylene group, a substituted or unsubstituted C2-C10 heteroarylene group, or a substituted or unsubstituted C4-C10 cycloalkylene group;
X₁ and X₂ are each independently a carboxylic acid group, a salt thereof, a sulfonic acid, a salt thereof, or an amino group; and
m is an integer between 1 and 10 wherein the self-dispersing colorant is in a range of 1 to 40 parts by weight based on 100 parts by weight of the liquid medium; and
filtering the self-dispersing colorant and the liquid medium.
